(19) United States
(12) Patent Application Publication
(10) Pub. No.: US 2006/0217602 A1
(43) Pub. Date: Sep. 28, 2006

(54) Method and apparatus for noninvasive targeting

(76) Inventors: Alan Abul-Haj, Mesa, AZ (US); Thomas B. Blank, Gilbert, AZ (US); Kevin H. Hazen, Gilbert, AZ (US); James Ryan Henderson, Phoenix, AZ (US); Timothy Ruchti, Gilbert, AZ (US); Josh Hope, Gilbert, AZ (US)

Correspondence Address: GLENN PATENT GROUP, 3475 EDISON WAY, SUITE L, MENLO PARK, CA, 94025, US

(21) Appl. No.: 11/367,884

(22) Filed: Mar. 3, 2006

Related U.S. Application Data
(60) Provisional application No. 60/658,821, filed on Mar. 4, 2005.

(51) Int. Cl. A61B 5/00

(52) U.S. Cl. 600/316; 600/310; 600/322

(57) ABSTRACT

The invention relates to noninvasive sampling. In one embodiment, the invention relates to a sample probe interface method and apparatus for targeting a tissue depth and/or pathlength that is used in conjunction with a noninvasive analyzer to control spectral variation. In a second embodiment, a signal from a sample or target probe of a tissue feature or volume is used in positioning a portion of a measuring system relative to the sample. The system is optionally used in conjunction with a targeting system used to control the sampling location of the measuring system.

### CROSS REFERENCE TO RELATED APPLICATIONS

This application claims:

priority to U.S. patent application Ser. No. 11/117,104 filed Apr. 27, 2005, which claims benefit of U.S. provisional patent application Ser. No. 60/566,568 filed Apr. 28, 2004; and

benefit of provisional patent application Ser. No. 60/658,821 filed Mar. 4, 2005.

### BACKGROUND OF THE INVENTION

1. Field of the Invention

The invention relates to noninvasive sampling. In one embodiment, the invention relates to a sample probe interface method and apparatus for targeting a tissue depth and/or pathlength that is used in conjunction with a noninvasive analyzer to control spectral variation. In a second embodiment, a signal from a sample or target probe of a tissue feature or volume is used in positioning a portion of a measuring system relative to the sample. The system is optionally used in conjunction with a targeting system used to control the sampling location of the measuring system.

2. Description of Related Art

Spectroscopy based noninvasive analyzers deliver external energy in the form of light to a sampling site, region, or volume of the human body where the photons interact with a tissue sample, thus probing chemical and physical features. Some of the incident photons are specularly reflected, diffusely reflected, scattered and/or transmitted out of the body where they are detected. A distinct advantage of a noninvasive analyzer is the analysis of chemical and structural constituents in the body without the generation of a biohazard and in a pain-free manner with limited consumables. Additionally, noninvasive analyzers allow multiple analytes or structural features to be determined at one time. Common examples of noninvasive analyzers are magnetic resonance imaging (MRI's), X-rays, pulse oximeters, and noninvasive glucose concentration analyzers. With the exception of X-rays, these determinations are performed with relatively harmless wavelengths of radiation.

Sampling Methodology

A wide range of technologies serve to analyze the chemical make-up of the body. These techniques are broadly categorized into two groups, invasive and noninvasive. Herein, a technology is referred to as invasive if the measurement process acquires any biosample from the body for analysis or if any part of the measuring apparatus penetrates through the outer layers of skin into the body. Noninvasive procedures do not penetrate into the body or acquire a biosample outside of their calibration and calibration maintenance steps.

Noninvasive Glucose Concentration Estimation

Diabetes is a chronic disease that results in abnormal production and use of insulin, a hormone that facilitates glucose uptake into cells. While a precise cause of diabetes is unknown, genetic factors, environmental factors, and obesity play roles. Diabetics have increased risk in three broad categories: cardiovascular heart disease, retinopathy, and neuropathy. Diabetics often have one or more of the following complications: heart disease and stroke, high blood pressure, kidney disease, neuropathy (nerve disease and amputations), retinopathy, diabetic ketoacidosis, skin conditions, gum disease, impotence, and fetal complications. Diabetes is a leading cause of death and disability worldwide. Moreover, diabetes is merely one among a group of disorders of glucose metabolism that also includes impaired glucose tolerance and hyperinsulinemia, which is also known as hypoglycemia. Long-term clinical studies demonstrate that the onset of diabetes related complications is significantly reduced through proper long-term control of blood glucose concentrations. Noninvasive glucose concentration estimation is projected by the inventors to aid in treatment of diabetics by facilitating more frequent glucose concentration estimations and hence proper long term control of diabetes mellitus.

There exist a number of noninvasive approaches for glucose concentration estimation in tissue or blood. These approaches vary widely but have at least two common steps. First, an apparatus is used to acquire a photometric signal from the body, typically without obtaining a glucose concentration estimation. Second, an algorithm is used to convert this signal into a glucose concentration estimation.

One type of noninvasive glucose concentration analyzer is a system performing glucose concentration estimations from spectra. Typically, a noninvasive apparatus uses some form of spectroscopy to acquire a signal, such as a spectrum, from the body. A particular range for noninvasive glucose concentration estimation in diffuse reflectance mode is in the near-infrared from approximately 700 to 2500 nm or one or more ranges therein, such as about 1100 to 2500 nm. These techniques are distinct from the traditional invasive and alternative invasive techniques in that the interrogated sample is a portion of the human body in-situ, not a biological sample acquired from the human body.

Calibration

Optical based glucose concentration analyzers require calibration. This is true for all types of glucose concentration analyzers, such as traditional invasive, alternative invasive, noninvasive, and implantable analyzers. A fundamental feature of noninvasive glucose analyzers is that they are secondary in nature, that is, they do not measure blood glucose concentrations directly. Therefore, a primary method is required to calibrate these devices to measure blood glucose concentrations properly. Many methods of calibration exist.

Instrumentation

There are a number of reports on noninvasive glucose technologies. Some of these relate to general instrumentation configurations required for noninvasive glucose concentration estimation while others refer to sampling technologies. Those related to the present invention are briefly reviewed here:

P. Rolfe, Investigating substances in a patient's bloodstream, U.K. patent application ser. no. 2,033,575 (Aug. 24, 1979) describes an apparatus for directing light into the body, detecting attenuated backscattered light, and using the collected signal to determine glucose concentrations in or near the bloodstream.

C. Dahne, D. Gross, Spectrophotometric method and apparatus for the non-invasive, U.S. Pat. No. 4,655,225 (Apr. 7, 1987) describe a method and apparatus for directing light into a patient's body, collecting transmitted or backscattered light, and determining glucose concentrations from selected near-infrared wavelength bands. Wavelengths include 1560 to 1590, 1750 to 1780, 2085 to 2115, and 2255 to 2285 nm with at least one additional reference signal from 1000 to 2700 nm.

R. Barnes, J. Brasch, D. Purdy, W. Lougheed, Non-invasive determination of analyte concentration in body of mammals, U.S. Pat. No. 5,379,764 (Jan. 10, 1995) describe a noninvasive glucose concentration estimation analyzer that uses data pretreatment in conjunction with a multivariate analysis to estimate blood glucose concentrations.

M. Robinson, K. Ward, R. Eaton, D. Haaland, Method and apparatus for determining the similarity of a biological analyte from a model constructed from known biological fluids, U.S. Pat. No. 4,975,581 (Dec. 4, 1990) describe a method and apparatus for measuring a concentration of a biological analyte, such as glucose concentration, using infrared spectroscopy in conjunction with a multivariate model. The multivariate model is constructed from a plurality of known biological fluid samples.

J. Hall, T. Cadell, Method and device for measuring concentration levels of blood constituents non-invasively, U.S. Pat. No. 5,361,758 (Nov. 8, 1994) describe a noninvasive device and method for determining analyte concentrations within a living subject using polychromatic light, a wavelength separation device, and an array detector. The apparatus uses a receptor shaped to accept a fingertip with means for blocking extraneous light.

S. Malin, G Khalil, Method and apparatus for multi-spectral analysis of organic blood analytes in noninvasive infrared spectroscopy, U.S. Pat. No. 6,040,578 (Mar. 21, 2000) describe a method and apparatus for determination of an organic blood analyte using multi-spectral analysis in the near-infrared. A plurality of distinct nonoverlapping regions of wavelengths are incident upon a sample surface, diffusely reflected radiation is collected, and the analyte concentration is determined via chemometric techniques.

Specular Reflectance

R. Messerschmidt, D. Sting Blocker device for eliminating specular reflectance from a diffuse reflectance spectrum, U.S. Pat. No. 4,661,706 (Apr. 28, 1987) describe a reduction of specular reflectance by a mechanical device. A blade-like device “skims” the specular light before it impinges on the detector. A disadvantage of this system is that it does not efficiently collect diffusely reflected light and the alignment is problematic.

R. Messerschmidt, M. Robinson Diffuse reflectance monitoring apparatus, U.S. Pat. No. 5,636,633 (Jun. 10, 1997) describe a specular control device for diffuse reflectance spectroscopy using a group of reflecting and open sections.

R. Messerschmidt, M. Robinson Diffuse reflectance monitoring apparatus, U.S. Pat. No. 5,935,062 (Aug. 10, 1999) and R. Messerschmidt, M. Robinson Diffuse reflectance monitoring apparatus, U.S. Pat. No. 6,230,034 (May 8, 2001) describe a diffuse reflectance control device that discriminates between diffusely reflected light that is reflected from selected depths. This control device additionally acts as a blocker to prevent specularly reflected light from reaching the detector.

Malin, supra describes the use of specularly reflected light in regions of high water absorbance, such as 1450 and 1900 nm, to mark the presence of outlier spectra wherein the specularly reflected light is not sufficiently reduced.

K. Hazen, G. Acosta, A. Abul-Haj, R. Abul-Haj, Apparatus and method for reproducibly modifying localized absorption and scattering coefficients at a tissue measurement site during optical sampling, U.S. Pat. No. 6,534,012 (Mar. 18, 2003) describe a mechanical device for applying sufficient and reproducible contact of the apparatus to the sampling medium to minimize specular reflectance. Further, the apparatus allows for reproducible applied pressure to the sampling site and reproducible temperature at the sampling site.

Temperature

K. Hazen, Glucose Determination in Biological Matrices Using Near-Infrared Spectroscopy, doctoral dissertation, University of Iowa (1995) describes the adverse effect of temperature on near-infrared based glucose concentration estimations. Physiological constituents have near-infrared absorbance spectra that are sensitive, in terms of magnitude and location, to localized temperature and the sensitivity impacts noninvasive glucose concentration estimation.

Pressure

E. Chan, B. Sorg, D. Protsenko, M. O'Neil, M. Motamedi, A. Welch, Effects of compression on soft tissue optical properties, IEEE Journal of Selected Topics in Quantum Electronics, Vol. 2, no. 4, pp.943-950 (1996) describe the effect of pressure on absorption and reduced scattering coefficients from 400 to 1800 nm. Most specimens show an increase in the scattering coefficient with compression.

K. Hazen, G. Acosta, A. Abul-Haj, R. Abul-Haj, Apparatus and method for reproducibly modifying localized absorption and scattering coefficients at a tissue measurement site during optical sampling, U.S. Pat. No. 6,534,012 (Mar. 18, 2003) describe in a first embodiment a noninvasive glucose concentration estimation apparatus for either varying the pressure applied to a sample site or maintaining a constant pressure on a sample site in a controlled and reproducible manner by moving a sample probe along the z-axis perpendicular to the sample site surface. In an additional described embodiment, the arm sample site platform is moved along the z-axis that is perpendicular to the plane defined by the sample surface by raising or lowering the sample holder platform relative to the analyzer probe tip. The '012 patent further teaches proper contact to be the moment specularly reflected light is about zero at the water bands about 1950 and 2500 nm.

Coupling Fluid

A number of sources describe coupling fluids with important sampling parameters.

Index of refraction matching between the sampling apparatus and sampled medium is known. Glycerol is a common index matching fluid for optics to skin.

R. Messerschmidt, Method for non-invasive blood analyte measurement with improved optical interface, U.S. Pat. No. 5,655,530 (Aug. 12, 1997), and R. Messerschmidt Method for non-invasive blood analyte measurement with improved optical interface, U.S. Pat. No. 5,823,951 (Oct. 20, 1998) describe an index-matching medium for use between a sensor probe and the skin surface. The index-matching medium is a composition containing perfluorocarbons and chlorofluorocarbons.

M. Robinson, R. Messerschmidt, Method for non-invasive blood analyte measurement with improved optical interface, U.S. Pat. No. 6,152,876 (Nov. 28, 2000) and M. Rohrscheib, C. Gardner, M. Robinson, Method and apparatus for non-invasive blood analyte measurement with fluid compartment equilibration, U.S. Pat. No. 6,240,306 (May 29, 2001) describe an index-matching medium to improve the interface between the sensor probe and skin surface during spectroscopic analysis. The index-matching medium is preferably a composition containing chlorofluorocarbons with optional added perfluorocarbons.

T. Blank, G. Acosta, M. Mattu, S. Monfre, Fiber optic probe guide placement guide, U.S. Pat. No. 6,415,167 (Jul. 2, 2002) describe a coupling fluid of one or more perfluoro compounds where a quantity of the coupling fluid is placed at an interface of the optical probe and measurement site. Perfluoro compounds do not have the toxicity associated with chlorofluorocarbons.

M. Makarewicz, M. Mattu, T. Blank, G. Acosta, E. Handy, W. Hay, T. Stippick, B. Richie, Method and apparatus for minimizing spectral interference due to within and between sample variations during in-situ spectral sampling of tissue, U.S. patent application Ser. No. 09/954,856 (filed Sep. 17, 2001) describe a temperature and pressure controlled sample interface. The means of pressure control are a set of supports for the sample that control the natural position of the sample probe relative to the sample.

Positioning

E. Ashibe, Measuring condition setting jig, measuring condition setting method and biological measuring system, U.S. Pat. No. 6,381,489, Apr. 30, 2002 describes a measurement condition setting fixture secured to a measurement site, such as a living body, prior to measurement. At time of measurement, a light irradiating section and light receiving section of a measuring optical system are attached to the setting fixture to attach the measurement site to the optical system.

J. Roper, D. Böcker, System and method for the determination of tissue properties, U.S. Pat. No. 5,879,373 (Mar. 9, 1999) describe a device for reproducibly attaching a measuring device to a tissue surface.

J. Griffith, P. Cooper, T. Barker, Method and apparatus for non-invasive blood glucose sensing, U.S. Pat. No. 6,088,605 (Jul. 11, 2000) describe an analyzer with a patient forearm interface in which the forearm of the patient is moved in an incremental manner along the longitudinal axis of the patient's forearm. Spectra collected at incremental distances are averaged to take into account variations in the biological components of the skin. Between measurements rollers are used to raise the arm, move the arm relative to the apparatus and lower the arm by disengaging a solenoid causing the skin lifting mechanism to lower the arm into a new contact with the sensor head.

T. Blank, G. Acosta, M. Mattu, S. Monfre, Fiber optic probe guide placement guide, U.S. Pat. No. 6,415,167 (Jul. 2, 2002) describe a coupling fluid and the use of a guide in conjunction with a noninvasive glucose concentration analyzer in order to increase precision of the location of the sampled tissue site resulting in increased accuracy and precision in noninvasive glucose concentration estimations.

T. Blank, G. Acosta, M. Mattu, M. Makarewicz, S. Monfre, A. Lorenz, T. Ruchti, Optical sampling interface system for in-vivo measurement of tissue, world patent publication no. WO 2003/105664 (filed Jun. 11, 2003) describe an optical sampling interface system that includes an optical probe placement guide, a means for stabilizing the sampled tissue, and an optical coupler for repeatably sampling a tissue measurement site in-vivo.

Raman

G. Lucassen, G. Puppels, P. Caspers, M. Van Der Voort, E. Lenderink, M. Van Der Mark, R. Hendricks, J. Cohen, Analysis of a composition, U.S. Pat. No. 6,609,015 (Aug. 19, 2003); G. Lucassen, R. Hendricks, M. Van Der Voort, G. Puppels, Analysis of a composition, U.S. Pat. No. 6,687,520 (Feb. 3, 2004); G. Lucassen, G. Puppels, M. Van Der Voort, Analysis Apparatus and Method, WIPO publication no. WO 2004/058058 (filed Dec. 4, 2003); F. Schuurmans, M. Van Beek, L. Bakker, W. Rensen, B. Hendricks, R. Hendricks, T. Steffen, Optical analysis system, WIPO publication no. WO 2004/057285 (filed Dec. 19, 2003); G. Lucassen, G. Puppels, M. Van Der Voort, R. Wolthuis, Apparatus and method for blood analysis, WIPO publication no. WO 2004/070368 (filed Jan. 19, 2004); R. Hendricks, G. Lucassen, M. Van Der Voort, G. Puppels, M. Van Beek, Analysis of a composition with monitoring, WIPO publication no. WO 2004/082474 (filed Mar. 15, 2004); M. Van Beek, C. Liedenbaum, G. Lucassen, W. Rensen Catheter head, WIPO publication no. WO 2004/093669 (filed Apr. 23, 2004); and M. Van Beek, J. Horsten, M. Van Der Voort, G. Lucassen, P. Caspers, Method and apparatus for determining a property of a fluid which flows through a biological tubular structure with variable numerical aperture, WIPO publication no. WO 2005/009236 (filed Jul. 26, 2004) describe a monitoring (targeting) system used to direct a Raman excitation system to a blood vessel.

The Raman spectroscopy targeting and imaging systems described above are fundamentally different than the vibrational absorption spectroscopy techniques taught herein. Raman spectra are obtained by irradiating a sample to a high energy state with a powerful source of visible monochromatic radiation. In addition, Raman spectroscopy is strongly dependent upon size of scattering particles. Finally, Raman signals require a change in polarizability of the probed molecule for a signal to be obtained. By contrast, infrared spectrometers use notably less intense broadband sources that do not excite molecules to high energy states. Further, infrared absorption spectroscopy in the region 1300 to 2500 nm is relatively insensitive to particle size. Finally, molecules that are vibrationally active require a change in dipole, which is associated with the vibrational mode of the molecule. This last point is critical. Raman signals require a change in polarizability while vibrational spectroscopy requires a change in dipole. This means molecular structure that is vibrationally infrared active is inactive in Raman spectroscopy and vise-versa. For example, near-infrared and infrared vibrational absorption spectroscopy show water to have very strong absorbance signal resulting in the primary interference in tissue analysis. By contrast, Raman signals are virtually unperturbed by water. As a result, in absorption spectroscopy regions of high water absorbance are necessarily avoided and penetration depths of photons into tissue from 1100 to 2500 nm is limited to millimeters. By contrast, Raman signals are possible in regions where water absorbs strongly in the infrared and a greater depth of penetration of photons into tissue is possible. Therefore, Raman and vibrational infrared spectroscopy operate under different theory, use different instrumentation, observe different molecular structure, and sample different tissue layers in skin.

To date, accurate and precise noninvasive analyte property estimations have not been generated in a reproducible fashion largely due to minimal signal to noise levels. A solution to the problem is to optimize resultant signal to noise by targeting analyte rich tissue volume or tissue layers with the probing photons. The method and apparatus results in increased precision and accuracy of noninvasive sampling and a means of assuring that the similar tissue sample volumes are repeatably sampled.

### SUMMARY OF THE INVENTION

The invention relates to noninvasive sampling. In one embodiment, the invention relates to a sample probe interface method and apparatus for targeting a tissue depth and/or pathlength resulting in enhanced signal of a noninvasive signal compared to a neighboring tissue volume. In a second embodiment, a signal from a sample or target probe of a tissue feature or volume is used in positioning a portion of a measuring system relative to the sample. The system increases precision and accuracy of sampling analyte rich tissue volume, which leads to improved accuracy and precision in noninvasive analyte property estimation. The invention is optionally used in conjunction with a targeting system used to direct a measuring system to a targeted sample site or volume.

### BRIEF DESCRIPTION OF THE DRAWINGS

FIG. 1 provides a block diagram of a measuring system according to the invention;

FIG. 2 illustrates control of depth of penetration and pathlength according to the invention;

FIG. 3 provides a block diagram of an analyzer with a targeting system and a measuring system according to the invention;

FIG. 4 is an example of an analyzer having a targeting system and a measuring system according to the invention;

FIG. 5 is a second example of an analyzer having a targeting system and a measuring system according to the invention;

FIG. 6 is a third example of an analyzer having a targeting system and a measuring system according to the invention;

FIG. 7 provides a block diagram of a two probe analyzer according to the invention;

FIGS. 8a and 8b illustrate an embodiment of a dynamic mount according to the invention; and

FIG. 9 provides a block diagram of processing spectra according to the invention.

### DETAILED DESCRIPTION OF THE INVENTION

Sampling is controlled to enhance analyte concentration estimation derived from noninvasive sampling. Based upon knowledge of the incident photons and detected photons, a chemical and/or structural basis of the sampled site is deduced. In a first embodiment of the invention, an analyzer controlling depth of penetration and/or optical pathlength of probing photons is used to estimate an analyte property. In one embodiment, the measuring system of an analyzer controls the pathlength and/or optical depth of the probing photons. In a second embodiment of the invention, a targeting system is used to direct a measuring system to a targeted tissue sample site or tissue volume. Either system increases analyte estimation performance by increasing precision and accuracy of sampling and/or by targeting an analyte rich tissue volume.

Examples provided herein are directed at noninvasive glucose concentration determination using near-infrared vibrational absorption spectroscopy. However, the principles widely apply to other noninvasive measurements and/or estimation of additional blood and/or tissue analytes using any form of spectroscopy. The examples are not intended to limit the invention to glucose concentration determination. In its broadest sense, the invention relates to noninvasive analyte property determination using spectroscopy.

Analyzer

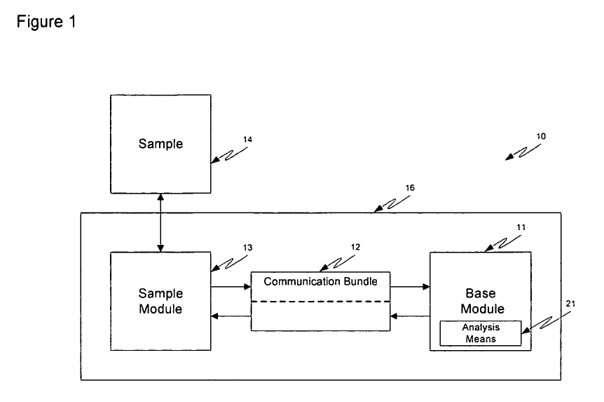

An analyzer includes a measuring system and optionally a targeting system. The measuring system is integral to the analyzer. The targeting system is optionally internal to the analyzer, semi-coupled to the analyzer, or is used separately from the analyzer in terms of time of use or in the space that is occupied. Herein, the combined base module 11, communication bundle 12, sample module 13, and processing center are referred to as a measuring system 16. The targeting system is described, infra. Referring now to FIG. 1, a block diagram of an exemplar measuring system 16 of the analyzer 10 is presented that includes a base module 11 and sample module 13 connected via communication means 12, such as integrated optics or a communication bundle. In addition, analysis means 21 are incorporated into the analyzer.

Coordinate System

Herein, an x, y, and z coordinate system relative to a given body part is defined. An x,y,z coordinate system is used to define the sample site, movement of objects about the sample site, changes in the sample site, and physical interactions with the sample site. The x-axis is defined along the length of a body part and the y-axis is defined across the body part. As an illustrative example using a sample site on the forearm, the x-axis runs between the elbow and the wrist and the y-axis runs across the axis of the forearm. Similarly, for a sample site on a digit of the hand, the x-axis runs between the base and tip of the digit and the y-axis runs across the digit. Together, the x,y plane tangentially touches the skin surface, such as at a sample site. The z-axis is defined as orthogonal to the plane defined by the x- and y-axes. For example, a sample site on the forearm is defined by an x,y plane tangential to the sample site. An object, such as a sample probe, moving along an axis perpendicular to the x,y plane is moving along the z-axis. Rotation or tilt of an object about one or a combination of axis is further used to define the orientation of an object, such as a sample probe, relative to the sample site.

Measurement System

Referring now to FIG. 1, a block diagram of a measurement system 16 of an analyzer 10 is provided. In one example, all of the components of the measuring system 16 of the noninvasive glucose analyzer 10 are included in a single unit, such as a handheld unit or a unit. In a second example, the measuring system 16 of the analyzer 10 is physically separated into elements, such as a base module in a first housing 11, a communication bundle 12, and a sample module in a second housing 13. Advantages of separate units include heat, size, and weight management. For example, a separated base module allows for support of the bulk of the analyzer on a stable surface, such as a tabletop or floor. This allows a smaller sample module to interface with a sample, such as human skin tissue. Separation allows a more flexible and/or lighter sample module for use in sampling by an individual. Additionally, separate housing requirements are achievable for the base module and sample module in terms of power, weight, and thermal management. In addition, a split analyzer results in less of a physical impact, in terms of mass and/or tissue displacement, on the sample site by the sample module. In a third example, the analyzer is a tabletop, rack, or wall mounted unit. In any embodiment, the communication bundle is optionally used to tether the sample module to the base module, is in the form or wireless communication, and/or is integrated into the analyzer, such as in a handheld version or in a single housing based analyzer. The sample module, base module, communication bundle, display module, processing center, and tracking system are further described, infra.

Either the integrated analyzer or split module analyzer is usable for personal monitoring, for chronic care, or for acute monitoring, such as in a nursing home, emergency room, critical care facility, medical professional building, intensive care unit, or daycare.

Pathlength/Depth of Penetration

Optionally, one or both of the targeting and measuring systems target a depth of skin tissue, a volume of tissue, and/or an optical pathlength, such as an average pathlength. In a first example, the measuring system is adjusted to a pathlength or depth in the absence of a targeting system. In a second example, the measuring system targets a net analyte signal. In a third example, a targeted depth is the cutaneous layer of skin tissue. Parameters that are used to control depth, average depth, distance to a targeted depth, pathlength within a targeted depth, or pathlength include any of incident angle of photons, distance between illumination and collection areas, size of the illumination and collection areas, numerical aperture of incident and/or collection optics, applied pressure to or about, the sample tissue, displacement of tissue, and mode of operation, such as transmittance, reflectance, or diffuse reflectance. Further, the apparatus and techniques used by the measuring system are optionally the same or variations of those taught for the targeting system, described infra.

The depth of penetration and pathlength of collected photons is dependent upon the tissue state and properties of the tissue, such as scattering and absorbance. Generally, lower scattering results in deeper maximum photon depth of penetration. As absorption increases, the photons traveling deeper have a smaller probability of returning to the incident surface. Thus, effective depth of penetration of collected photons is dependent upon scattering and absorbance. In addition, scattering and depth of penetration affect the optical pathlength. Generally, photons collected at an incident surface with deeper penetration and/or greater diffusion have, on average, longer pathlengths. Because scattering and absorbance are wavelength dependent, the average depth of penetration and pathlength are also wavelength dependent. Hence, techniques that alter any of the above mentioned properties are optionally used to target a depth in the tissue, such as applied pressure to a sample site or altering the sample site temperature.

Generally, the analyte signal or net analyte signal of the analyte property is preferably maximized by controlling a number of parameters, such as signal, noise, interference, and sampling. One method of optimizing these parameters is by photonically sampling a tissue volume rich in the analyte of interest. In the case of a hydrophilic analyte, such as glucose, depth targeting of photons into an aqueous rich layer increases the sampling photon density in the analyte rich region and minimizes photon density in analyte poor regions, such as the adipose layer. A number of exemplary instrument configuration examples are provided for optimizing the analyte signal, infra.

### EXAMPLE I

In a first example, a cutaneous sampling optical probe uses a distance between incident photons directed at the skin and the collected photons coming from the skin to control average depth of penetration and/or average optical pathlength of the probing photons. Optional distances include a minimum distance, a maximum distance, and both a minimum and maximum distance. For example, very short pathlengths are effectively blocked using a distance, such as about 0.1, 0.3, 0.5, 0.7, 1, 2, or 3 mm, between a region of incident photons contacting the skin and a region where photons are collected from the skin. Blocking photons is accomplished by a number of means including use of any of a thin or thick blocker, such as a blade, a gap, a spacer, and an optically opaque sheath, such as a fiber optic coating. This spacer is optionally used to block specular light in embodiments where the optics contact or come into close proximity with the skin. A maximum range is defined by the far reaches between the incident illumination area and collection area.

### EXAMPLE II

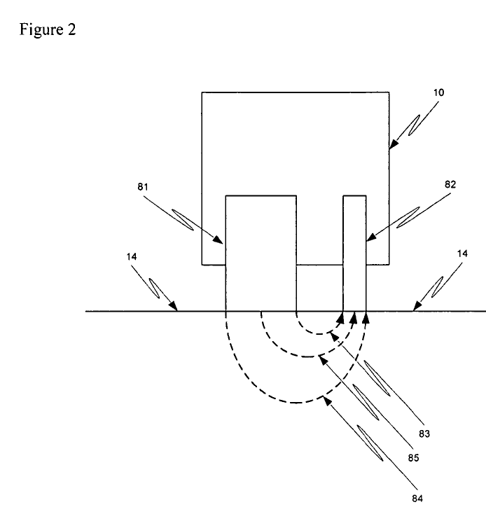

Referring now to FIG. 2, an example of an analyzer 10 with a given pathlength and/or optical depth is provided. An illumination probe 81 delivers photons to the tissue sample 14. A collection probe 82 collects light emerging from a collection area. On average, short depths and short photonic pathlengths in tissue result from photons with the shortest distance to travel 83. Photons having the longest distance to travel between the illumination area and collection area typically have the largest average depth of penetration and pathlength 84. Intermediate distances typically result in intermediate depths of penetration and pathlength 85. The average pathlength and depth of penetration is increased by moving the illumination area further from the collection area. Similarly, smaller pathlengths and shallower penetration depths are achieved by moving the illumination area closer to the collection area. By controlling the illumination area(s), collection area(s), sizes and locations of the either area, and distance(s) between the areas, the sample volume, optical depth, and pathlength are controlled in a manner that enhances or optimizes the net analyte signal. Optionally, an optical signal representative of the sample site is used in controlling parameters that affect the probed sample volume and/or optical pathlength. For example, an optical signal is obtain and used in a closed-loop system for directing movement of one or more analyzer components, to guide sampling of the system to an analyte rich sample volume, pathlength, or depth. In one case, pathlength is controlled in order to enhance subsequent chemometric methods that rely on a fixed or narrowly defined pathlength of the probing photons.

### EXAMPLE III

In a third example of the invention, a fiber bundle or a plurality of bundlets are used to control the analyte signal through controlling variables, such as pathlength and/or depth of penetration. The spacing between the illumination and collection fibers of each bundlet, and the spacing between bundlets is optimized to minimize sampling of the adipose subcutaneous layer and to maximize collection of light that has been backscattered from the cutaneous layer. This example optimizes penetration depth by limiting the range of distances between illumination fibers and detection fibers. By minimizing sampling of the adipose layer, interference contributed by the fat band is greatly reduced in the sample spectrum, thereby increasing the signal-to-noise ratio for the target analyte. In addition, by maximizing photonic sampling of an aqueous rich layer, such as the dermis, analyte signal for hydrophilic analytes, such as glucose, are enhanced. Similarly, maximizing photonic sampling of adipose rich regions optimizes signal of hydrophobic analytes, such as cholesterol. The provision of multiple bundlets also minimizes interference in the sample spectrum due to placement errors.

### EXAMPLE IV

In another example, mechanical and/or optical means are used to change the illumination area of a sample optically probed by a source and/or the collection area observed by a detector. As described herein, this changes the average pathlength and depth of penetration. For example, a changing blocker thickness or iris diameter is used to expand or contract the illumination and/or detector area or move the average distance between an incident photon and a collection photon area. For the case of the blocker, the illumination area and/or collection area are moved so as to change the distance between the areas. In a first case, the area is changed by mechanically moving a probe part or optic. In a second case, an iris is used in the optical path that is widened or narrowed as a function of time. In the first case, a wider aperture in the illumination system increases the tissue area illuminated. If the detection area is unchanged, this decreases the average collected photon distance between the illumination and detection area with resultant changes in the probed tissue volume. The iris is optionally mechanically opened or closed or is optionally optically widened or narrowed with the use of a liquid crystal. The iris need not be round. Rather, the iris is optionally of any shape acting to partially close along an x, y, or x and y axis like a shutter.

### EXAMPLE V

In a fifth example, an analyzer uses a liquid crystal to block, make opaque, or make transparent one or more parts within the optical train of the analyzer. The liquid crystal transmittance is controlled by charge placed into the crystal or a current flowing about the crystal. By adjusting the transmittance of various areas, such as illumination and/or collection area, the sampled tissue volume is changed. For example, illumination and/or collection areas interfacing to a tissue sample are varied. In one case, an optic interface with the skin is used to block varying distances between the illumination and collection areas as a function of time. When the blocked area increases the distance between the illumination and collection areas, the observed pathlength and depth of penetration of the sampling photons in the tissue sample increases. The transmittance of the liquid crystal controlled regions is optionally wavelength dependent. In a first case, the optional wavelength dependence allows short pathlengths for highly absorbing wavelengths and longer pathlengths for wavelengths that absorb less. In a second case, the wavelength dependence is used to remove undesirable wavelengths of light in a manner acting as a longpass, shortpass, or bandpass filter.

### EXAMPLE VI

In a sixth example, a reflector shape is changed with time causing the illumination area lit or detection area observed to expand or contract. For example, a shape of a back reflector behind a source is changed to create larger or smaller illumination areas, such as a circle with a different diameter, on the sample. For example, a flexible substrate is reflective coated and used as the backreflector. In addition to changing the illuminated area, the angle of incidence of the illumination photons contacting the skin is changed. This alters the tissue volume that is probed.

### EXAMPLE VII

In a seventh example, the incident angle of the photons is changed. This alters the initial angle of the photons entering the sample. This initial angle operates in conjunction with scattering and absorbance to result in an altered average depth of penetration and/or pathlength of the photons into the sample.

### EXAMPLE VIII

In another example, the illumination light is brought to the sample by a group of illumination fibers contacting the sample, proximately contacting the sample, or not contacting the sample. Light is collected from the sample by one or more detection fibers. The illumination fibers are located at different distances from or different distributions of distances from the detection fiber(s). To modify or control the pathlength groups of illumination fibers at different distances from the detection fiber are filled with light. The resultant detected light has different sampled pathlengths and depths.

In a slightly modified approach, either the illumination and/or detection fibers are angled relative to one another or to the skin. For example, the detection fiber(s) are in contact with the sample and the illumination fibers present light while their angles are varied or controlled to maximize or minimize a detected analyte signal. The analyte signal is optionally provided as feedback to actively control the fiber angles or to actively control any positioning or orientation of any analyzer component.

Another modified approach fills the illumination fibers with different wavelengths so that an analyte signal is controlled. For example, illumination fibers near the detection fiber are filled with wavelengths of light that are more highly absorbed by the sample and can therefore only travel a short path. Illumination fibers more distant from the detection fiber are filled with light that is absorbed very little and can therefore travel longer paths through the sample. The penetration depths are manipulated by the same mechanisms.

### EXAMPLE IX

In an additional example, the pathlengths and/or the penetration depths are controlled by moving a mask with openings that allow the light to pass onto the sample at specific positions and/or at specific times. In one configuration, the mask is a rotating disc with a series of openings of various sizes and locations. The disc is preferably located between the light source and the sample. The rotation of the disc causes the locations and areas of illumination to be controlled. For example a wheel is rotated in the optical train prior to the sample. The wheel has transmissive, semi-transmissive, or opaque regions as a function of wavelength and/or position. In the case of a wheel with open sections and closed sections that is spun, the average pathlength is varied dependent upon the location of the openings. The wheel is spun in a light source that the average distance of the open areas varies as a function of time. Preferably a second wheel is used so that only the open areas of interest are viewed at a given time by the detecting system. This allows a detector to see different depths of the same sample through time or for an array to see different depths and pathlengths of a sample at a single point in time or through time.

### EXAMPLE X

In yet another example, part of an analyzer is redirected to a new sample site as a function of time or based upon collected and analyzed noninvasive signal in real-time or pseudo-real time. For example, part of an analyzer or sample probe is aimed at a new sample area. For example, an actuator is used to move a beam directing optic and/or sample probe to illuminate a new sample area. Similarly, an actuator is used to move a light collection optic to observe a different sample tissue area and/or volume.

### EXAMPLE XI

In a further example of the invention, a real-time or quasi-real time signal is collected, optionally processed, and fed back to a controller that adapts the analyzer to the sample as a function of time. This allows an intelligent system to lock in on a signal and adjust hardware and/or software parameters as a function of time to maximize the observed analyte signal.

### EXAMPLE XII

In yet another example of the invention, illumination of an area is achieved through the use of a fiber optic. When source radiation is fed normally into a fiber, the fiber optic yields a Gaussian distribution of photon density across the opposite end of the fiber in terms of a given cross section. This results in most of the light being emitted by the fiber at the center of the fiber core. Alternatively, the fiber is loaded with source photons at an angle. This results in a bimodal distribution of photon intensity emission from the end of the fiber as a cross sectional view. This results in photon density being relatively larger near the outer edge of the fiber. As a result, the photon density across the exit end of the fiber is a function of the loading angle of the source radiation into the fiber. In addition, the photons emitted from the end of fiber into tissue have different pathlengths to a collection area dependent upon where the photons exited the fiber. This allows the loading angle of photons into the fiber to sample different tissue volumes. Therefore, means to control sample fiber optic loading are used to control sampling depth, pathlength, and tissue volume thereby affecting the net analyte signal.

### EXAMPLE XIII

In still yet another embodiment of the invention, incident light is sent into the tissue at varying angles. The angle at which the incident light enters the tissue alters the probed tissue volume, as measured by parameters including depth of penetration, average pathlength, and analyte signal-to-noise ratio. Therefore, means for controlling incident light angles affect which probed tissue volume is observed and the detected analyte signal.

### EXAMPLE XIV

In a further example of the invention, a bead or layer of fluid is placed onto tissue. A collection optic, such as a tip of a fiber optic, is immersed into the fluid. Incident light is preferably directed at the skin tissue in regions outside of the fluid or bead of fluid. This allows an analyzer with minimal contact with a tissue sample. Controllable variables in this embodiment include: the type of fluid, an optional coating on the fluid, the distance of a collection fiber relative to the skin surface, the angle of the fiber end relative to the skin surface, the distance from the fluid at which incident photons hit the skin. Each of these variables affect the observed tissue volume and net analyte signal observed. Optionally, incident photons penetrate through the contacting fluid exclusively or inclusively of incident photons penetrating skin outside of the contacting fluid.

### EXAMPLE XV

In yet another embodiment of the invention, probed tissue pathlength is controlled by tailoring the distance distribution between optical illuminator conduits and the detector conduit using a digital mirror array. In this embodiment, light passes from a multiplicity of illuminator conduits into the skin and from the skin into a centrally located detector conduit. Source light is separated into different optical channels defined by individual fibers in a short fiber bundle into which the source light is focused. Preferably, a digital mirror array, or DLP chip, is used to separate the source light into individual fibers or a few fibers in an illumination bundle. Focused light is reflected off of the mirror array onto the fiber optic and individual mirror angles on the chip are controlled to reflect full, partial, or no intensity onto individual illumination fibers. Since each fiber represents an element in the source/detector distance distribution, manipulation of the reflected light allows for tailoring or even optimization of the light launch distribution into the tissue. Such flexibility allows for pathlength control or correction of the measured diffuse reflectance signal.

### EXAMPLE XVI

In yet another example of the invention, the measuring system target is any of:
a natural tissue component;
a chemical feature;
a physical feature;
an abstract feature;
a marking feature added to the skin;
a skin surface feature;
a measurement of tissue strain;
tissue morphology;
a target below the skin surface;
a manmade target;
a fluorescent marker;
a subcutaneous feature;
a dermal layer;
a dermis thickness within a specification;
capillary beds;
a capillary;
a blood vessel; and
a subcutaneous layer.

Permutations and combinations of the above described examples are possible as are permutations and combinations of the examples with the apparatus and techniques describe in the targeting, analyzer, and processing sections herein.

Measuring System/Targeting System

In another embodiment of the invention, a measurement system is used in conjunction with a targeting system. In one example, the targeting system identifies the sample in terms of x-, and y-position. The measuring system then samples primarily at a given depth z, as described herein. In a second example, the targeting system targets a sample in at least one of x-, y-, and z-position and optionally in terms of sample probe tilt and/or rotation. In the second example, a measurement system is directed to the targeted sample or a sample position spatially related to the targeted position at the same or different time with a common or separate sample probes as described, infra. The targeted signal is optionally a spectral indicator of average depth. An example targeted signal is a signal related to a constituent present in a hydrophilic volume growing relative to signal related to constituents in hydrophobic regions. An additional example is a signal using an absorbance dominated signal and/or a scattering dominated signal.

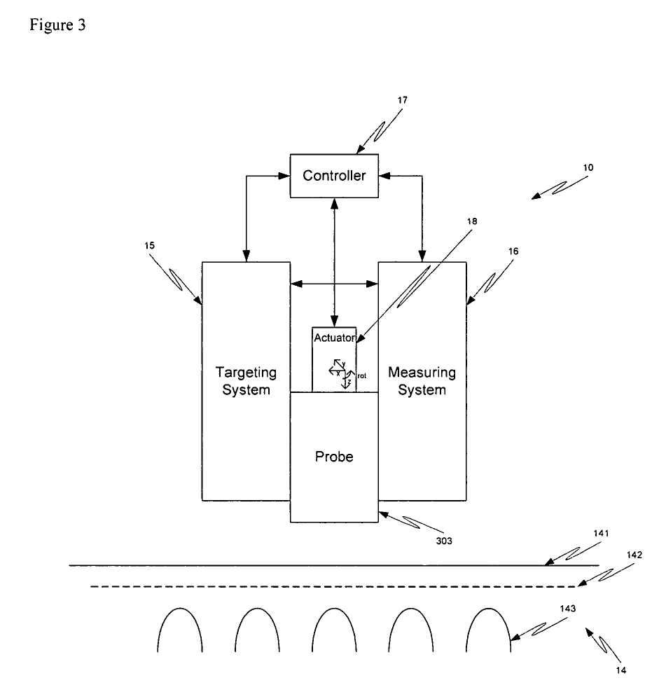

Referring now to FIG. 3, a block diagram of an analyzer 10 is presented that is configured with two primary systems, a targeting system 15 and a measuring system 16. The targeting system targets a tissue area or volume of the sample 14. For example, the targeting system targets a surface feature 141, one or more volumes or layers 142, and/or an underlying feature 143, such as a capillary or blood vessel. The measuring system contains a sample probe 303, which is optionally separate from or integrated into the targeting system. The sample probe of the measuring system is preferably directed to the targeted region or to a location relative to the targeted region either while the targeting system is active or subsequent to targeting. Less preferably, use of the measuring system is followed by use of the targeting system and a targeting image is used to post process the measuring system data. A controller 17 is used to direct the movement of the sample probe 303 in at least one of the x-, y-, and z-axes via one or more actuators 18. Optionally the controller directs a part of the analyzer that changes the observed tissue sample in terms of surface area or volume. Optionally, the controller moves a fixture holding the sample relative to the analyzer. The controller communicates with the targeting system, measuring system, and/or controller.

There exist a large number of targeting and measuring system configurations. In its broadest sense, targeting signal is acquired as a function of time and position and used to position the measuring system. The targeting signal is from the targeting system or from the measuring system. Several exemplar embodiments are provided, infra. Some features of the configurations are outlined here. The targeting system and measuring system optionally use a single source that is shared or have separate sources. The targeting is optionally used to first target a region and the measuring system subsequently samples at or near the targeted region. Alternatively, the targeting and measuring system are used over the same period of time so that targeting is active during sampling by the measuring system. The targeting system and measuring system optionally share optics and/or probe'the same tissue area and/or volume. Alternatively, the targeting and measuring system use separate optics and/or probe different or overlapping tissue volumes. In various configurations, neither, one, or both of the targeting system and measuring system are brought into contact with the skin tissue 14 at or about the sample site. Each of these parameters are further considered, infra. Finally, permutations and combinations of the strategies and components of the embodiments presented herein are possible.

The targeting system targets a target. Targets include any of:
a natural tissue component;
a chemical feature;
a physical feature;
an abstract feature;
a marking feature added to the skin;
a skin surface feature;
a measurement of tissue strain;
tissue morphology;
a target below the skin surface;
a manmade target;
a fluorescent marker;
a subcutaneous feature;
a dermis thickness within a specification, such as about 0.5, 1, 2, 3, 4, or 5 millimeters thick;
capillary beds;
a capillary;
a blood vessel; and
arterial anastomoses.

Examples of marking features added to the skin include a tattoo, one or more dyes, one or more reflectors, a crosshair marking, a manmade marking element, and positional markers, such as one or more dots or lines. Examples of a skin surface feature include a wart, hair follicle, hair, freckle, wrinkle, and gland. Targeted tissue morphology includes surface shape of the skin, such as curvature and flatness. Examples of specifications for a dermis thickness include a minimal thickness and a maximum depth. For example, the target is a volume of skin wherein the analyte, such as glucose, concentration is higher. In this example, the measuring system is directed to a state probing photons at a depth of the enhanced analyte concentration.

Targeting System

A targeting system targets a target. A targeting system typically includes a controller, an actuator, and a sample probe that are each described infra. Examples of targeting systems include a planarity detection system, optical coherence tomography (OCT), a proximity detector and/or targeting system, an imaging system, a multiple detector system, a two-detector system, and a single detector system. Examples of targeting system technology include: capacitance, impedence, acoustic signature, ultrasound, use of a pulsed laser to detect and determine distance, and the use of an electromagnetic field, such as radar and high frequency radio-frequency waves. Sources of the targeting system include a laser scanner, ultrasound, and light, such as ultraviolet, visible, near-infrared, mid-infrared, and far-infrared light. Detectors of the targeting system are optionally a single element, a two detector system, an imaging system, or a detector array, such as a charge coupled detector (CCD) or charge injection device or detector (CID). One use of a targeting system is to control movement of a sample probe relative to a sample site or location. A second example of use of a targeting system is to make its own measurement. A third use is as a primary or secondary outlier detection determination. In its broadest sense, one or more targeting systems are used in conjunction with or independently from a measurement system.

Different targeting techniques have different benefits. As a first example, mid-infrared light samples surface features to the exclusion of features at a depth due to the large absorbance of water in the mid-infrared. A second example uses the therapeutic window in the near-infrared to image a feature at a depth within tissue due to the light penetration ability from 700 to 1100 nm. Additional examples are targeting with light from about 1100 to 1450, about 1450 to 1900, and/or about 1900 to 2500 nm, which have progressively shallower penetration depths of about 10, 5, and 2 mm in tissue, respectively. A further example is use of visible light for targeting or imaging greater depths, such as tens of millimeters. Still an additional example is the use of a Raman targeting system, such as in WIPO international publication number WO 2005/009236 (Feb. 3, 2005), which is incorporated herein in its entirety by this reference thereto. A Raman system is capable of targeting capillaries. Multiple permutations and combinations of optical system components are available for use in a targeting system.

Controller

A controller controls the movement of one or more sample probes of the targeting and/or measuring system via one or more actuators. The controller optionally uses an intelligent system for locating the sample site and/or for determining surface morphology. For example, the controller hunts in the x- and y-axes for a spectral signature. In a second example, the controller moves a sample probe via the actuator toward or away from the sample along the z-axis. The controller optionally uses feedback from the targeting system, from the measurement system, or from an outside sensor in a closed-loop mechanism for deciding on targeting probe movement and for sample probe movement. In a third example, the controller optimizes a multivariate response, such as response due to chemical features or physical features. Examples of chemical features include blood/tissue constituents, such as water, protein, collagen, elastin, and fat. Examples of physical features include temperature, pressure, and tissue strain. Combinations of features are used to determine features, such as specular reflectance. For example, specular reflectance is a physical feature optionally measured with a chemical signature, such as water absorbance bands centered at about 1450, 1900, or 2600 nm. Controlled elements include any of the x-, y-, and z-axes positions of sampling along with rotation or tilt of the sample probe. Also optionally controlled are periods of light launch, intensity of light launch, depth of focus, and surface temperature. In a fourth example, the controller controls elements resulting in pathlength and/or depth of penetration variation. For example, the controller controls an iris, rotating wheel, backreflector, or incident optic, which are each described supra.

Tissue Strain

The controller optionally moves the targeting probe and/or sample probe so as to make minimal and/or controlled contact with the sample to control stress and/or strain on the tissue, which is often detrimental to a noninvasive analyte property estimation. Strain is the elongation of material under load. Stress is a force that produces strain on a physical body. Strain is the deformation of a physical body under the action of applied force. In order for an elongated material to have strain there must be resistance to stretching. For example, an elongated spring has strain characterized by percent elongation, such as percent increase in length.

Skin contains constituents, such as collagen, that have spring-like properties. That is, elongation causes an increase in potential energy of the skin. Strain induced stress changes optical properties of skin, such as absorbance and scattering. Therefore, it is undesirable to make optical spectroscopy measurements on skin with various stress states. Stressed skin also causes fluid movements that are not reversible on a short timescale. The most precise optical measurements would therefore be conducted on skin in the natural strain state, such as minimally or non-stretched stretched skin. Skin is stretched or elongated by applying loads to skin along any of the x-, y-, and z-axes, described infra. Controlled contact reduces stress and strain on the sample. Reducing stress and strain on the sample results in more precise sampling and more accurate and precise glucose concentration estimations. An example of using light to measure a physical property, such as contact,

stress, and/or strain, in tissue is provided. Incident photons are directed at a sample and a portion of the photons returning from the sample are collected and detected. The detected photons are detected at various times, such as when no stress is applied to the tissue and when stress is applied to the tissue. For example, measurements are made when a sample probe is not yet in contact with the tissue and at various times when the sample probe is in contact with the tissue, such as immediately upon contact and with varying displacement of the sample probe into the tissue. The displacement into the tissue is optionally at a controlled or variable rate. The collected light is used to determine properties. One exemplary property is establishing contact of the sample probe with the tissue. A second exemplary property is strain. The inventors determined that different frequencies of light are indicative of different forms of stress/strain. For example, in regions of high water absorbance, such as about 1450 nm, the absorbance is indicative of water movement. Additional regions, such as those about 1290 nm, are indicative of a dermal stretch. The time constant of the response for water movement versus dermal stretch is not the same. The more fluid water movement occurs approximately twenty percent faster than the dermal stretch. The two time constants allow interpretation of the tissue state from the resultant signal. For example, the interior or subsurface hydration state is inferred from the signal. For example, a ratio of responses at high absorbance regions and low absorbance regions, such as about 1450 and 1290 nm, is made at one or more times during a measurement period. Changes in the ratio are indicative of hydration. Optionally, data collection routines are varied depending upon the determined state of the tissue. For example, the probing tissue displacement is varied with change in hydration. The strain measurement is optionally made with either the targeting system or measurement system. The tissue state probe describe herein is optionally used in conjunction with a dynamic probe, described infra.

Actuator

An actuator moves the sample probe relative to the tissue sample. One or more actuators are used to move the sample probe along one or more of the x-, y-, and z-axes. In addition, the tilt of the sample probe relative to the xy-plane tangential to the tissue sample is optionally controlled. The targeting system preferably operates in conjunction with the measurement system, described, supra.

Targeting and Measurement System

The benefits described, supra, for controlling pressure, stress, and/or strain on the sample by controlling the movement of the targeting system sample probe relative to the tissue also apply to controlling the movement of the measurement system sample probe.

### EXAMPLE XVII

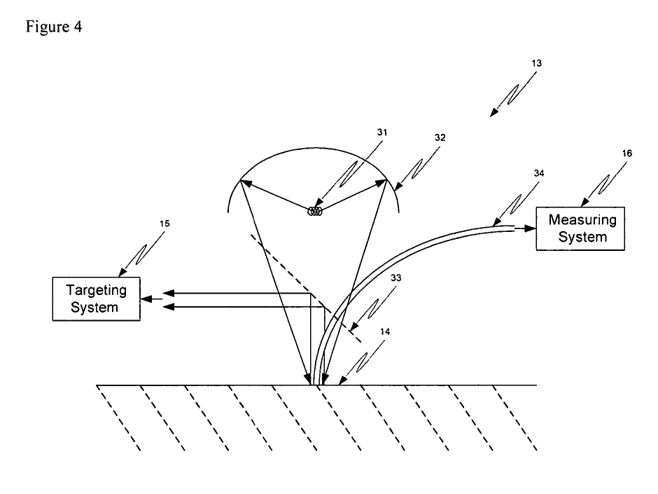

In still an additional embodiment of the invention, a measurement system is used in conjunction with a targeting system. The targeting system preferably finds an x, y-location for sampling. Alternatively, the targeting system is used to target at least one of the x-, y-, and z-positions for sampling. In this example, a single source is used for the targeting system and the measuring system. Alternatively separate sources are used in the targeting and measuring system. Referring now to FIG. 4, an illustrative sample module 13 portion of an analyzer 10 is presented. Within the sample module, photons from source 31 are directed to a sample 14 either directly or via one or more optics, such as a back reflector 32 or a lens. In one case, the incident photons pass through a dichroic filter 33. A portion of the incident photons either reflect off of the surface or are diffusely reflected from a volume of the tissue sample 14. A portion of the specular and/or diffusely reflected photons are directed to a targeting system 15. In this example, the collection optics uses a dichroic filter 33 that reflects a portion of the specular or diffusely reflected to the targeting or imaging system 15. In this example, a collection optic 34, such as a fiber optic, is used to collect diffusely reflected photons. The end of the fiber optic is preferably in close proximity to the surface of the tissue sample 14. The housing or casing of the fiber optic is preferably used to block specularly reflected light, as described infra. The collected light is directed to the remainder of the measuring system 16. Optionally, coupling fluid is used at the sample module 13 skin tissue 14/interface. This example is illustrative of a system that uses a single source for the targeting system and measuring system. In addition, this example is illustrative of a system where the targeting system is used to target a sample prior to measurement or at the same time of operation of the measurement system. Still further, this example is illustrative of a targeting system that images substantially the same volume that the measuring system observes.

### EXAMPLE XVIII

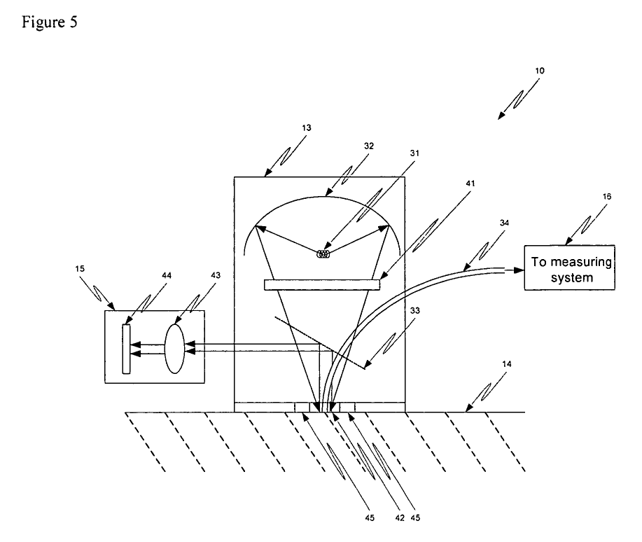

Referring now to FIG. 5, yet another example of the invention is provided. A sample module 13 portion of an analyzer 10 is presented. A source 31 emits light. At least part of the emitted light is incident upon a sample tissue site 14. In this example, a backreflector 32 focuses a portion of the emitted light 31 through an optional first optic 41, through an optional second optic 42, and optionally through a fluid coupler. The incident photons are optionally controlled by an aperture defined by an outer radial distance of a incident light blocker. A portion of the incident photons penetrate into the sample 14 where they are transmitted, scattered, diffusely reflected, and/or absorbed. A portion of the photons in the sample exit the sample site 14 and are directed to the targeting system 15 or measuring system element 16. In the case of the targeting system 15, light is optionally directed via optics or mirrors 43 to a detector array 44. In the case of the measuring system 16, light is collected with one or more collection optics 34, such as a fiber optic. An optional guide element 45 or mount element is used to control the positioning of the incident photons.

In multiple embodiments of the invention, a first optic and a second optic are used in the optical path between the source element 31 and the tissue sample 14.

First Optic

An optional first optic 41 is placed in the optical path after the source element 31 and preferably before the tissue sample 14. In its broadest sense, the first optic includes at least one of the following parameters: optically passes desirable wavelengths of light, optically blocks at least one region of undesirable wavelengths of light, limits radiative heat transmitted to the tissue sample, and is not in contact with the tissue sample.

The first optic passes desirable wavelengths of light, such as about 1200 to 1850 nm, or sub-regions therein, such as about 1300 to 1700 nm. Within the transmissive region, high transmittance, such as greater than ninety percent, is desirable, but any transmittance is acceptable as long as sufficient analyte signal is achieved. The first optic is, optionally, anti-reflective coated or is index of refraction matched to adjoining surfaces in the optical path. In some embodiments, the first optic also passes light used for imaging, such as a region in the visible or in the near-infrared from about 700 to 1100 nm.

The first optic preferably blocks or strongly diminishes light throughput in at least one undesirable spectral region emitted by the source or entering through ambient conditions. For example, the first optic is used to remove unwanted ultraviolet (UV), visible (VIS), and/or near-infrared light from about 700 to 1000 nm. Optionally, light of having longer wavelengths than the spectral region collected and analyzed is removed in order to remove unwanted heat resulting from photon flux onto the sample and to reduce heating of optics later in the optical path. Photons removed by the filter that result in the heating of the filter do not result in direct heating of the sample site via radiative heating or photonic heating. Rather, the much slower and less efficient conduction or convection processes convey this heat. This reduces the risk of over heating the skin.

Second Optic

A second optic 42 is optionally placed in the optical path after the source element 31 and before the tissue sample 14. In its broadest senses, the second optic passes desirable wavelengths of light and/or optically blocks at least one region of undesirable wavelengths of light. The, optional, second optic is in close proximity to the tissue sample. This allows control of radiative and/or conductive heat transmitted to the tissue sample and or control of specular reflectance as described, infra.

The second optic 42 is, optionally, used to control thermal transfer to the tissue sample. In one embodiment of the invention, the second optic is of low thermal conductivity. The low thermal conductivity minimizes conductive heating of the sample by the raised temperature of the sample module 13 due to heating by the source. Examples of low thermal conductivity materials that are transmissive in the spectral region of interest include, silica, Pyrex™, sapphire, and some glasses and plastics. Optionally, the second optic has higher thermal conductivity and is used to more rapidly adjust the tissue sample 14 temperature to that of the tissue sample contacting area of the sample module 13. An example of a higher thermally conductive material is silicon. The second optic optionally surrounds a detector or a detection optic 34, such as a fiber. In one case, the second optic provides mechanical support to the fiber optic and aids in positioning the collection fiber in close proximity to the sample and/or to aid in reduction of collection of specularly reflected light by blocking the light with the cladding, buffer, coating, or surrounding material of the fiber optic. An optional spacer is provided between the fiber core and the incident photons. The fiber coating and/or spacer provide specular reflectance blocking and/or depth of penetration and pathlength control as described, infra. The maximum penetration of the photons into the tissue sample preferably exceeds the radial dimension of the spacer.

### EXAMPLE XIX

In a another example of the invention, the measuring system is used as a targeting system. The measuring system, in this example, has targeting system capabilities. The measuring system is used to both target the sample and to subsequently or concurrently measure the sample. A separate targeting system is not needed in this example.

### EXAMPLE XX

In one embodiment of the invention, the targeting system or measurement system uses capacitance sensors or touch sensors for determining any of:
tilt of a sample probe relative to a sample site;
distance of a sample probe tip to a sample site;
x,y-position of a sample probe tip relative to a sample site;
relative distance of a sample probe tip to a sample site; and
contact of a sample probe tip with a sample site.

For a capacitance based targeting system, capacitance, C, is calculated according to Equation 1

$$C ∝ \frac{A}{d} \qquad ( 1 )$$

where capacitance, C, is proportional to the area, A, of the capacitor divided by the distance, d, between the capacitor plates. The capacitor has two plates. The first capacitor plate is integrated or connected to the measuring system, such as at the sample module and preferably at the sample module sample probe tip. The second capacitor is the deformable material, such as a skin sample, body part, or a the tissue sample site. The assumption is that the person is a capacitor. A typical adult has a capacitance of about 120 pF. The time constant of a capacitor/resistor is calculated according to Equation 2
T=RC (2)
where the time constant, T, is equal to the resistance, R, times the capacitance, C. Hence, the distance between the capacitor plates is calculated through the combination of equations 1 and 2 through the measurement of the circuit time constant. For example, the time constant is the time required to trip a set voltage level, such as about 2.2 volts, given a power supply of known power, such as about 3.3 volts. The time constant is used to calculate the capacitance using equation 2. The capacitance is then used to calculate the distance or relative distance through Equation 1. For example, as a distance between a sample site, such as a forearm or digit of a hand, and the capacitor plate decreases, the time constant increases and the capacitance increases. The measure of distance is used in positioning the probe at or in proximate contact with the sample site without disturbing the sample site.

In use, the distance or relative distance between the sample probe tip and the sample site is determined, preferably before the tip of the sample probe displaces localized sample site skin/tissue, which can lead to degradation of the sample integrity in terms of collected signal-to-noise ratios and/or sampling precision. Examples are used to illustrate the use of the capacitance sensor in the context of a noninvasive analyte property determination.

In one example, the distance or relative distance between the sample probe tip and the sample site is determined using a single capacitor. The sample probe is brought into close proximity with the sample site using the time constant/distance measurement as a metric. In this manner, the sample probe is brought into close proximity to the sample site without displacing the sample site. Due to the inverse relationship between capacitance and distance, the sensitivity to distance between the sample site and the sample probe increases as the distance between the sample probe and the sample site decreases. Using capacitance sensors, the distance between the sample site and the tip of the sample probe is readily directed to a distance of less than about one millimeter. Capacitance sensors as used herein are also readily used to place the sample probe tip with a distance of less than about 0.1 millimeter to the sample site. In this example, multiple capacitors are optionally used to yield more than one distance reading between the sample probe tip and the sample site. Multiple capacitive sensors are optionally used to control tilt along x- and/or y-axes.

In a second example, two or more capacitance sensors are optionally used for leveling the tip of the sample probe relative to the morphology of the sample site. The distance between the sample site and the probe tip is measured using two or more capacitor pairs. For example, if one capacitor reads a larger distance to the sample site than the second capacitor, then the probe tip is moved to level the probe by moving the larger distance side toward the sample, the smaller distance side away from the sample, or both. The sample probe tip tilt or angle is either moved manually or by mechanical means.

### EXAMPLE XXI

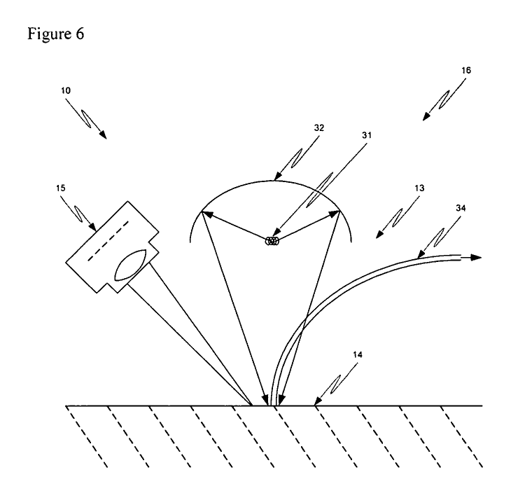

Referring now to FIG. 6, an example of a separate targeting system and sampling system is presented. The targeting system 15, such as a camera system or endoscope, targets a first site or volume. The measuring system 16 targets a second site or volume. The two sample sites optionally overlap, partially overlap, or are separated. Preferably, the first site and second site overlap so that the targeted site is the site sampled. Alternatively, the first site is separated from the second site. The controller is used to adjust a sample probe of the measuring system relative to the targeted volume or area. This allows the targeting system to find and target one feature and the measuring system to measure a separate feature.

In this example, the targeting system and measuring system have separate sources and optical trains. Additionally, in this example the targeting system is used before and/or concurrently in time with the measuring system.

### EXAMPLE XXII

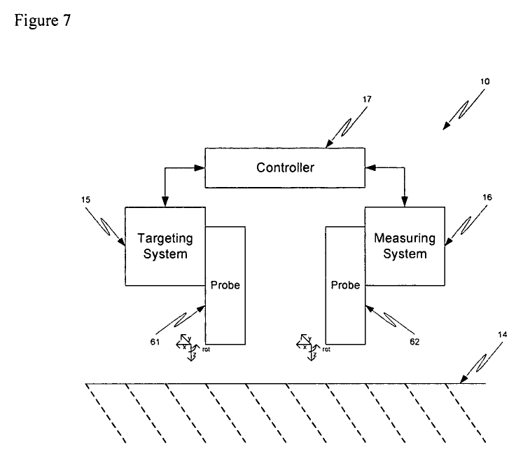

Referring now to FIG. 7, an example of an analyzer 10 with two separate sample probes is presented. A first sample probe 61 is part of a targeting system 15. A second sample probe 62 is part of a measuring system 16. The sample probes 61, 62 each are independently controlled via one of more controllers 17. The sample probes move along any of the x-, y-, and z-axes and each have optional rotation and/or tilt control. The sample probes 61, 62 are used at the same or different times. The sample probes sample different tissue sample 14 volumes or the same tissue sample volume at different times. The two sample probes 61, 62 move in synchronization or are moved independently of each other.

Analyzer

In one embodiment of the invention, the apparatus and/or measuring system control pathlength and/or depth of penetration. Optional components and/or controls of the apparatus include any of:
a targeting system;
an adaptive sample probe head;
a dynamic sampling probe;
a specular reflectance blocker;
occlusion and/or tissue hydration control;
a coupling fluid;
an automated coupling fluid delivery system;
a guide;
a mount;
a system for reducing stress/strain on the tissue;
a system for controlling skin tissue state;
a split system;
a system for reducing and/or controlling thermal changes of the skin tissue;
means for minimizing sampling error;
an intelligent system for data processing;
a basis set; and/or
a data processing algorithm.

The split system, depth control, pathlength control, and targeting system are described, supra. Each of the remaining components, processes, algorithms, or controls are briefly described, infra.

Adaptive Probe

The targeting system and/or measuring system are optionally controlled in at least one of x-, y-, and z-axes and optionally in rotation or tilt. This allows the probing system to adapt to the skin tissue surface. In this case, the sample probe is an adaptive probe with the benefit of reducing stress/strain upon sampling, as described supra.

An adaptive sample probe of the targeting and/or measuring system positions the corresponding sample probe tip at varying positions relative to a tissue sample. As the state of the skin changes, the adaptive probe adjusts the position of the sample probe tip or imaging interface relative to the tissue sample site. A first characteristic of the adaptive mount is achievement of highly repeatable sampling by limiting stress and strain on and/or about the median targeted tissue measurement site. In this manner, the skin undergoes minimal stress as the skin is not deformed to force the exact same position of the tissue to be sampled with each measurement. This leads to enhanced sampling precision and hence better accuracy and precision of one or more determined analyte properties.

An additional benefit of an adaptive probe is that it optionally provides a means for locally registering the location of the targeted and or measured tissue volume with respect to the optical probe and/or tip of a sample module, such that a narrow range of tissue volumes are sampled by the optical system(s). Local registration refers to controlling the position of the optical probe relative to a target and/or measurement location of the tissue. The adaptive probe allows flexibility in terms of the exact position of the tissue that is sampled. Means for registering the sample probe to the tissue are preferably optical, but are optionally mechanical and/or electromechanical.

Dynamic Sampling Probe

The sample probe is optionally used in a dynamic manner. For example, the targeting system sample probe 61, the measuring system sample probe 62, and/or a shared sample probe 303 are optionally dynamic. A dynamic probe is moved in a controlled fashion relative to a tissue sample in order to control spectral variations resulting from the sample probe displacement of the tissue sample during a sampling process.

A noninvasive analyzer 10 controls movement of a dynamic sample probe along any of the x-, y-, and z-axes and optionally controls tilt and/or rotation of the sample probe relative to a sampled tissue 14. in a first case, a dynamic probe facilitates positioning of the sample probe prior to data collection. In a second case, the ability to move the sample probe relative to the tissue sample as a function of time allows a dynamic tissue measurement. A dynamic tissue measurement is designed to collect time serial spectral data that contains the dynamic tissue response of the tissue sample as the sample probe is brought into contact with the tissue sample. In this measurement process spectral raster scans are collected continuously or semi-continuously as the sample probe is moved into contact with the tissue sample and/or as the sample probe displaces the tissue sample. For example, the sample probe is lowered slowly onto the targeted measurement site with or without an optical probe placement guide while the instrument acquires signal. In one case, a sample probe is controlled at least along the z-axis perpendicular to the x,y plane tangential to the surface of the sampled site thereby controlling displacement of the sample probe relative to a sample. The z-axis control of the displaced sample probe element of the sample module provides for collection of noninvasive spectra with a given displacement of a tissue sample and for collection of noninvasive spectra with varying applied displacement positions of the sample probe relative to the nominal plane of the sample tissue surface.

Specular Reflectance

The interface between the optical probe and the skin surface at the tissue measurement site is potentially a significant source of sampling error due to:
skin state change;
skin deformation with time;
skin stress/strain;
temperature mismatch;
lost dynamic range of detection system;
air gaps; and
refractive index mismatch.

These issues are distinct, but have some interrelationships. Incident light normal to the surface penetrates into the skin sample based upon the difference in refractive index, Snell's Law. For the refractive index of skin, approximately 1.38, and the refractive index of air, approximately 1.0, approximately four percent of the normally incident light is reflected and ninety-six percent of the light penetrates into the skin if the surface is smooth. In practice, the rough tissue surface results in an increased percentage of specularly reflected light. In addition, the percentage of light penetrating into skin varies as the index of refraction of the interfacing material to skin changes. Further, the coupling changes with the use of an intermediate material, such as water or a coupling fluid.

The amount of light that is specularly reflected is determined to degrade noninvasive estimations of low signal to noise analytes. A targeting or measuring sample probe that does not contact the surface of the skin, is not proximate the skin, and/or is not coupled to the skin via a coupling fluid, results in specular reflectance off of the diffusely reflecting skin surface that is partially caught in collection optics. This specular reflectance is difficult to remove once in the collection optic system and it is subsequently observed with the detector system. The specular signal is often much larger in magnitude across the desired spectral region compared with the analyte signal. For example, four-percent specular light is orders of magnitude larger than a noninvasive glucose signal from the glucose molecule that is present in about the 30 to 600 mg/dL range. It is therefore beneficial to have an optical system that removes the specular component. One method for removing specular light is to have part of the sample probe contact the skin surface. For example, having an optically opaque part contact the skin between the incident and collection photons forces the collected photons to have gone through at least a portion of the skin. Examples of specular blockers include a thin or thick blade blocker or a fiber optic cladding or buffer. One or both of the targeting system and measurement system optionally has a specular blocker.

Measurement Site Occlusion/Hydration

An optional aspect of the optical sampling system used in combination with one or both of the targeting and measurement system is the maintenance of an optimal level of hydration of the surface tissue at the measurement site for enhancement of the optical signal, sample reproducibility, and suppression of surface reflectance. Skin hydration means are optionally used with the targeting and/or measuring system. Skin surface irregularities result in an increase in surface reflection of the incident light. Surface irregularities of skin mean that the incident light is not normal to the surface. This results in more reflected light, and less penetrating light. In addition, air gaps in the outer layers of skin result in more reflected light that does not penetrate to the analyte containing region. A fraction of the light penetrating into an outermost layer of skin hits one or more air pockets and is reflected off of each surface of the air pocket. Many air pockets or poor hydration lead to a significant reduction in the percentage of incident photons that penetrate through the outermost skin layers, such as the stratum corneum, to the inner skin layers. Increasing the hydration of the outermost layers of skin decreases the impact of air pockets on the incident signal. Hydration, thus, results in a greater percentage of the incident photons reaching analyte rich skin volume. Hydration is achieved through a variety of means, such as occlusion, direct water contact, and increasing localized perfusion.

A preferred means of the optional hydration step is hydration by occlusive blockage of trans-epidermal water loss. This blockage ensures a steady state hydration as water diffusing from interior tissue is trapped in the stratum corneum. Attainment of high hydration levels reduces the water concentration gradient that provides the driving force for this trans-epidermal water movement. In a first case, an occlusive plug fits snugly into a guide aperture during periods between measurements, acting to insulate the tissue in the guide aperture from trans-epidermal water loss and the environmental effects of temperature and humidity that are known to influence the stratum corneum hydration state. In a second case, an occlusive patch is used, such as wrapping or covering the tissue sample site with a flexible polymer sheet. In a third case, a window or optic is contacted with the sample site to increase the localized skin surface and shallow depth hydration and/or to stabilize the tissue by providing the same tissue displacement as the probe. The optic is continuously, replaceably, or intermittently attached to the sample site. Examples of optics include a window, a longpass filter, and a bandpass filter. In a fourth case, hydration means include a material that provides a hydration barrier, thus promoting full and stable hydration of the stratum corneum. Typically, the occlusion means use a hydrophobic material, such as cellophane. In general, optional perfusion enhancement or regulation means are used to increased precision and accuracy in analyte property estimation by the removal or reduction of dry or pocketed skin at the sampling site.

Coupling Fluid

A coupling fluid is optionally used with the targeting and/or measuring system. An optical coupling fluid with a refractive index between that of the skin surface and the contacting medium is preferably used. However, a coupling fluid need not be a refractive index matching fluid in order to increase light throughput. For example, in the case of a high refractive index material, such as a lens, optical window, or filter, coming into contact with skin via a coupling fluid, the coupling fluid need not have a refractive index between that of skin and the optic to be beneficial. For example, the percentage of incident photons passing through a silicon lens into skin is increased even with use of a coupling fluid that does not have a refractive index between that of silicon and skin. For example, FC-40 (a fluorocarbon) has an index of refraction of 1.290 that is not between that of skin, 1.38, and silicon, approximately 3.45. However, the FC-40 still increases incident photon penetration by displacement of air. For example, for coupling silicon and skin FC-40 is not an “index-matching medium”, optical coupling fluid, or refractive-index matching coupling fluid. However, FC-40 is a coupling fluid that aids in light coupling by displacing the air.

Preferable coupling fluids are minimally inactive or inactive in terms of absorbance in the spectral region of interest. For example, in the near-infrared fluorocarbons, such as FC-40, have minimal absorbance and are good coupling fluids. In addition, coupling the relatively smooth surface of an optical probe with the irregular skin surface leads to air gaps between the two surfaces. The air gaps create an interface between the two surfaces that adversely affects the measurement during optical sampling of tissue due to refractive index considerations as described, supra. A coupling medium is used to fill these air gaps. Preferably, for an application, such as noninvasive glucose estimation, the coupling fluid:
is spectrally inactive;
is non-irritating;
is nontoxic;
has low viscosity for good surface coverage properties;
has poor solvent properties with respect to leaching fatty acids and oils from the skin upon repeated application; and
is thermally compatible with the measurement system.

In one example, a coupling fluid is preheated to between about 90 and 95° F., preferably about 92° F. Preheating the coupling fluid minimizes changes to the surface temperature of the contacted site, thus minimizing spectral changes observed from the sampled tissue.

Mount

In the preferred embodiment of the invention, neither the targeting system nor the measurement system use a mount in the sampling process. However, a guide or optionally a mount is optionally used with one or both of the targeting system and measurement system.

A key characteristic of an optional adaptive mount is achievement of highly repeatable sampling by limiting stress and strain on and/or about the median targeted tissue measurement site. To achieve this, the mount adapts to physical changes in the sample. An additional benefit of the adaptive mount is that it optionally provides a means for locally registering the location of the targeted tissue volume with respect to the optical probe and/or tip of a sample module, such that a narrow range of tissue volumes are sampled by the optical system. Local registration refers to controlling the position of the optical probe relative to a target location on the tissue. The adaptive mount allows flexibility in terms of the exact position of the tissue that is sampled. This allows the sample to undergo stress, expand, contract, and/or twist and the mount adapts to the new state of the sample by mounting a sample probe to a slightly new position in terms of x-position and y-position, described infra. Means for registering the guide and the optical probe are optionally mechanical, optical, electrical, and/or magnetic.

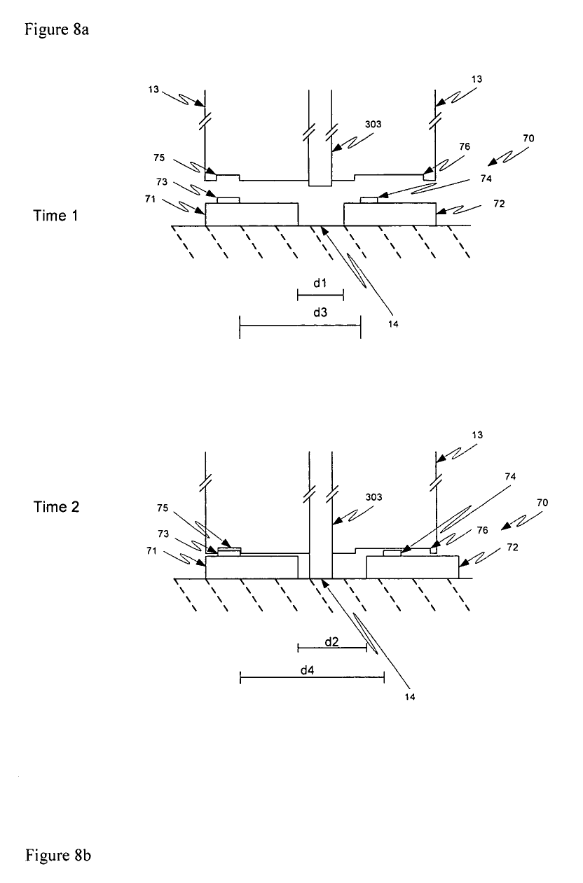

An example of an adaptive mount is presented that increases precision and accuracy of noninvasive sampling, which results in increased sensitivity, precision, and accuracy of subsequent analyte property estimation derived from the sampling. The adaptive mount is placed onto the skin of a person. Between uses, opposing ends of the adaptive mount move relative to each other as the skin tissue state changes. During use, the adaptive mount is designed to minimize skin deformation during placement of a sample probe of an analyzer or during placement of a plug. In one example, the adaptive mount samples a dynamic x-, y-position at or about a central sample site. In another example, the adaptive mount is deformable, which distributes applied forces during sample about the sample site. In these examples, at least one axis of the sample probe is allowed to float relative to a fixed x,y-point that defines a given sample site. Referring now to FIGS. 8a and 8b, an example of an adaptive mount with freedom of motion along the x-axis is presented at two moments in time.

At time 1(FIG. 8a), the tissue 14 has a distance, dl, between a first alignment piece 71 and a second alignment piece 72. The two alignment pieces 71, 72 have corresponding means for registration 73, 74. The two registration pieces 73, 74 pieces are integral to the alignment pieces 71,72 or are separate pieces. At time 1, the two registration pieces 73, 74 have a distance, d3, between them.

In this case, the registration pieces protrude from the alignment pieces. A portion of a sample module 13 is represented near the tissue 14. Registration pieces 75, 76 correspond to the registration pieces on the mount 73, 74, respectively. A sample probe 303 is situated at a given x-, y-position relative to the tissue 14.

At time 2 (FIG. 8b), the tissue 14 has changed state. In the state pictured, the tissue has elongated resulting in the distance between the first and second alignment pieces 71, 72 to expand in distance from d1 to d2. The corresponding distance between the first and second registration pieces 73, 74 has similarly expanded in distance from d3 to d4. In the example, the sample module 13 includes one registration piece 75 that couples with a corresponding registration piece 71 on the mount 70. A second registration piece 76 on the sample module 13 has freedom of movement in at least one-dimension relative to the alignment piece 72 and/or registration piece 74. The tip of the sample probe 303 mounts to a slightly different x-, y-position of the tissue 14 as the tissue state changes in a manner that effects the tissue size, shape, and or torque. This results in at least a portion of the sample module 13 and/or sample probe 303 to mount on the mount 70 via one or more alignment pieces and/or one or more registration pieces with minimal deformation or strain on the tissue 14. The mounting of the sample probe 303 to the mount 70 with minimal strain results in noninvasive spectra with fewer spectral interferences and hence corresponding analyte property estimation is more precise and accurate. Optionally, the sample probe 303 is movable along the z-axis, so that the tip of the sample probe results in minimal stress on the sample tissue volume. In the pictured instance, the sample probe is shown as extended to the tissue 14 at time 2. A movable z-axis sample probe is optionally used with this system, supra. Similarly, the variable placement of the sample probe relative to the tissue is performed along the y-axis or through a combination of x- and y-axis.

Data Processing

Chemometrics is the application of statistical and mathematical methods to physical measurement data. Chemometric techniques presented herein include pre and post signal processing and multivariate regression. Data preprocessing and/or data processing techniques are optionally used in combination with the invention. Generally, a method and apparatus correct for tissue related interference for the purpose of calibration and measurement of biological parameters noninvasively. The method is described in terms of outlier identification; filtering, such as use of a derivative; spectral correction; and baseline subtraction steps that, when used together, enable the noninvasive measurement of biological parameters, such as glucose.

Preprocessing

The tissue measurement optionally undergoes a preprocessing step to enhance the analytical signal and attenuate noise. Preprocessing comprises any of such techniques as:
referencing;
converting to absorbance;
filtering;
normalizing;
wavelength selection; and
performing a translation operation.

The choice of preprocessing techniques is dependent at least in part on the source of the analytical signal. Following preprocessing, a preprocessed tissue measurement is passed to the next step. If preprocessing has been omitted, the unprocessed tissue measurement is passed to the next step.

Feature Extraction

Feature extraction is optionally used in data analysis. Feature extraction is any mathematical transformation that enhances a quality or aspect of the sample measurement for interpretation. The general purpose of feature extraction is to concisely represent or enhance any of the structural, chemical physiological, and optical properties of the tissue measurement site that are related to the target analyte. For the purposes of the invention, a set of features is developed that is indicative of the effect of the target analyte on the probed tissue. The set of features represents or reflects tissue properties or characteristics that change in various ways according to changes in the any of the structural, chemical, physical, and physiological state of the tissue. The invention advances the state of current technology through extraction of features that represent changes in the physical state, chemical state, and/or physiological properties or characteristics of the tissue from a prior state.

The features targeted for extraction directly represent the analyte or indirectly represent the analyte, such as through tissue properties related to:

1) the concentration of water in each of the compartments;

2) the relative concentration of water in the compartments;

3) the size of the various compartments;

4) the change in electrical impedance resulting from the redistribution of water; and

5) the change in radiation emanating from the tissue.

Given the tissue measurement:
simple features are derived directly from the tissue measurement;
additional (derived) features are determined from the simple features through one or more mathematical transformation such as addition, subtraction, division, and multiplication; and
abstract features are derived through linear and nonlinear transformations of the tissue measurement.

While simple and derived features generally have a physical interpretation related to the properties of the tissue, such as the magnitude of the fat absorbance, the set of abstract features does not necessarily have a specific interpretation related to the physical system. For example, the scores of a factor analysis, principal component analysis, or partial-least squares decomposition are used as features, although their physical interpretation is not always known. The utility of the principal component analysis is related to the nature of the tissue measurement. The most significant variation in the tissue measurement is not caused directly by glucose but is related to the state, structure, and composition of the measurement site. This variation is modeled by the primary principal components. Therefore, the leading principal components tend to represent variation related to the structural properties and physiological state of the tissue measurement site and, consequently, reflect the tissue properties.

Several examples of extracted features are presented, which are illustrative of the optional feature extraction step. In a first example, for the case of noninvasive measurement of glucose through near-infrared spectroscopy, a resolved estimate of the magnitude of the fat band absorbance is used to infer specific information about the dermis. Although fat is relatively absent from the dermis, near infrared radiation must propagate through the dermis to penetrate the adipose tissue beneath. Thus, physiological changes or changing a targeted depth by the analyzer lead to corresponding changes in the optical properties of the dermis that influence the level of near-infrared radiation that penetrates to and is absorbed by the fat in adipose tissue. Therefore, the magnitude of the fat band present in a near-infrared absorbance spectrum varies, in part, according to the variation in the optical properties of the dermis. For example, as the water concentration in the dermis increases, the detected magnitude of the fat band naturally decreases and vice versa. Additional examples of features include a glucose absorbance bands at about 1590, 1730, 2150, or 2272 nm; a water absorbance band centered at about 1450, 1900, or 2600 nm; a fat absorbance band centered at about 1675, 1715, 1760, 2130, 2250, or 2320; and/or a protein absorbance band centered at about 1180, 1280, 1690, 1730, 2170, or 2285. Additional examples of extracted features include physical light scattering related information at wavelengths shorter than about 1300 nm; and/or chemical absorbance information at wavelengths longer than about 1300 nm.

Processing

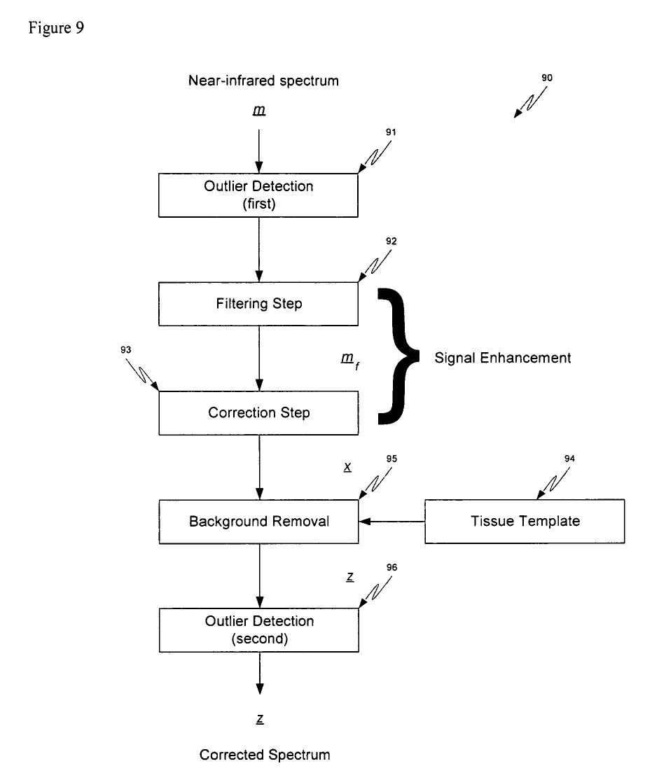

Referring now to FIG. 9, a block diagram summarizing one embodiment of processing 90 of the near-infrared signal is presented. The steps are all preferably used in the order illustrated. Alternatively, one or more steps are omitted and/or the steps are performed in alternative order. The method optionally includes both gross 91 and detailed 96 methods for detecting outliers or anomalous measurements that are incompatible with the processing methods or are the result of sampling or instrument errors. Spectral correction, involving the steps of filtering 92 and/or correction 93, is applied to compensate for noise and interference and to adjust a spectrum according to local or minor changes in the optical properties of the tissue sample. The step of background removal 95 reduces variation in the measurement, such as variation associated with sample-site differences, dynamic tissue changes, and subject-to-subject variation. An optional tissue template 94 is used to remove background 95. Examples of a tissue template include a spectrum of the subject being measured, a basis set, or a computed spectrum of a cluster.

A background removal step preferably follows the steps defined above and uses a spectral background or tissue template. For example, the background removal step performed by calculating the difference between the estimated spectral background or tissue template and x through
z=x−(cxₜ+d) (3)
where xₜ is the estimated background or tissue template, c and d are slope and intercept adjustments to the tissue template. Direct subtraction is just one form of background removal. The spectrally corrected signal, z, is used for calibration development or measurement of a target analyte. The background is estimated on the basis of an optimal selection of spectrally corrected measurements collected prior to the measurement, m. The variables c and d are preferably determined on the basis of features related to the dynamic variation of the tissue. In one embodiment, xₜ is a spectrally corrected spectral measurement collected on tissue at the beginning of a measurement period. The measurement period is defined as a time period during which the state of the tissue sample is uniform.

In a first example, the background removal step uses a basis set of spectral interferences to remove the signals that are specific to a given sampled tissue volume, such as the background. The optical estimate of the background are performed subsequent to the removal of noise and the correction of the spectrum. If this operation were implemented prior to spectral correction, signal components detrimental result in the spectrum that compromise the estimate of the background and lead to degraded results.

In a second example, the following steps are performed to process the spectra:
averaging spectra;
correcting dead pixels;
calculating absorbance;
performing x-axis standardization;
uniformly re-sampling the spectrum to standardize the x-axis;
performing a first (gross) outlier detection;
correcting the spectrum;
performing a wavelength selection;
removing interference; and
performing a second (fine) outlier detection.

The order of the steps is optionally varied. For example, the wavelength selection step is optionally performed out of sequence, such as after the second outlier detection or before any of the earlier steps. In addition, not all steps are required. For example, correcting dead pixels is not appropriate to some analyzers. As a second example, conversion to absorbance is not always required, nor are other steps. Multivariate analysis is optionally used after preprocessing. The data analysis means are optionally contained in a data processing module contained in the analyzer, such as in the base module.

Intelligent System

An optional intelligent system for measuring blood analyte properties is used in combination with the invention. The system operates on near-infrared absorbance spectra of in-vivo skin tissue. The architecture employs a pattern classification engine to adapt the calibration to the structural properties and physiological state of the patient as manifested in the absorbance spectrum. Optionally, a priori information about the primary sources of sample variability are used to establish general categories of patients. Application of calibration schemes specific to the various categories results in improved calibration and prediction.

Two classification methods are optionally used. The first assumes the classes are mutually exclusive and applies specific calibration models to the various patient categories. The second method uses fuzzy set theory to develop calibration models and blood analyte predictions. In the second, each calibration sample has the opportunity to influence more than one calibration model according to its class membership. Similarly, the predictions from more than one calibration are combined through defuzzification to produce the final blood analyte property estimation.

Based on the two classification rules, two implementations of the intelligent measurement system are detailed for the noninvasive estimation of the concentration of blood glucose. The first uses spectral features related to gross tissue properties to determine which of several prediction models is the most likely to produce an accurate blood glucose estimation. The extracted features are representative of the actual tissue volume irradiated. The second employs a fuzzy classification system to assign a degree of membership in each of several classes to the spectral measurement. The membership is used to aggregate the predictions of calibrations associated with each class to produce the final blood glucose prediction. Optionally, the membership strategy is employed during calibration in modified form of weighted principal components regression to produce calibrations from the entire population of samples.

In still yet an additional embodiment of the invention, a measurement system is used for both measurement and for targeting. In this embodiment of the invention, the measurement system has integrated into it any of the techniques taught herein for the targeting system and is used to target any of the targets taught herein for the targeting system.

Those skilled in the art will recognize that the present invention may be manifested in a variety of forms other than the specific embodiments described and contemplated herein. Departures in form and detail may be made without departing from the spirit and scope of the present invention. Accordingly, the invention should only be limited by the Claims included below.

1. A method for noninvasively determining an analyte property of a tissue sample, comprising the steps of:
collecting a feedback targeting signal with a targeting system;
adjusting an optical measuring system to enhance sampling of at least a tissue layer, said tissue layer having an analyte concentration exceeding an average concentration of said analyte in all of said tissue sample, wherein said step of adjusting uses said feedback targeting signal;
noninvasively collecting at least one spectrum of said tissue sample using said adjusted optical measuring system; and
chemometrically determining said analyte property from said at least one spectrum.

2. The method of claim 1, wherein said targeting signal comprises a signal representative of at least one of:
a target below a surface of said tissue sample;
a subcutaneous feature; and
a dermis thickness within a specification.

3. The method of claim 1, wherein said targeting system comprises at least one capacitance sensor.

4. The method of claim 1, wherein said targeting system comprises use of any of:
an imaging system;
a multiple detector system;
an impedence reading;
an acoustic signature;
an ultrasound system;
a pulsed laser; and
an electromagnetic field.

5. The method of claim 4, wherein said targeting signal comprises a signal representative of any of:
a manmade target;
a natural tissue component;
a chemical feature;
a physical feature;
an abstract feature of a skin parameter;
a fluorescent marker;
a marking feature added to the skin;
a skin surface feature;
a tissue morphology; and
a measurement of tissue strain.

6. The method of claim 1, wherein said step of adjusting positions said measuring system in terms of any of:
an x-axis position;
a y-axis position;
a z-axis position; and
tilt of a sample probe of said measuring system relative to a surface of said tissue sample.

7. The method of claim 1, wherein said step of adjusting changes at least one of orientation, shape, and position of an internal optical component of said measuring system resulting in change of direction of photons projecting from said analyzer.

8. The method of claim 1, wherein said step of adjusting controls an average depth of penetration into said tissue sample for detected photons.

9. The method of claim 1, wherein said step of adjusting targets an average optical pathlength for detected photons.

10. The method of claim 1, wherein said tissue layer comprises an aqueous based layer when said analyte is hydrophilic.

11. The method of claim 1, wherein said tissue layer comprises an adipose rich layer.

12. The method of claim 1, wherein said analyte comprises a blood borne constituent.

13. The method of claim 1, wherein said step of determining said analyte property through use of chemometrics comprises use of at least one of:
multivariate analysis; and
a derivative.

14. An apparatus for noninvasively determining glucose concentration, comprising:
a spectroscopic analyzer comprising a targeting system and a measuring system;
means for collecting a feedback signal of a skin tissue with said targeting system;
means for adjusting said measurement system to enhance targeting of an internal tissue layer;
means for collecting a second signal representative of at least said tissue layer using said adjusted measurement system; and
a chemometrics module for deriving said glucose concentration from said second signal.

15. The apparatus of claim 14, wherein said feedback signal comprises a signal representative of at least one of:
a target below a surface of said skin tissue;
a subcutaneous feature; and
a dermis thickness within a specification.

16. The apparatus of claim 14, wherein said targeting system comprises at least one capacitance sensor.

17. The apparatus of claim 14, wherein said means for adjusting repositions said measuring system in terms of any of:
an x-axis position;
a y-axis position;
a z-axis position; and
tilt of a sample probe of said measurement system relative to a surface of said skin tissue.

18. The apparatus of claim 14, wherein said step of adjusting changes at least one of orientation, shape, or position of an internal optical component of said analyzer resulting in change of direction of incident photons projecting from said analyzer.

19. The apparatus of claim 14, wherein said step of adjusting controls an average depth of penetration into said tissue sample for detected photons.

20. The apparatus of claim 14, wherein said step of adjusting targets an average optical pathlength for detected photons.
